(19) United States
(12) Patent Application Publication
(10) Pub. No.: US 2010/0330175 A1
(43) Pub. Date: Dec. 30, 2010

(54) CROSS-LINKED POLYALLYLAMINE TABLET CORE

(76) Inventor: Christopher N. Jobdevairakkam, Plainsboro, NJ (US)

Correspondence Address: ST. ONGE STEWARD JOHNSTON & REENS, LLC, 986 BEDFORD STREET, STAMFORD, CT, 06905-5619, US

(21) Appl. No.: 12/491,046

(22) Filed: Jun. 24, 2009

(51) Int. Cl. A61K 9/20; A61K 9/28; A61K 31/765; A61P 13/12

(52) U.S. Cl. 424/465; 424/474; 424/78.37

(57) ABSTRACT

A method and a composition for making a composition, tablet, or tablet core having cross-linked polyallylamine salts such as sevelamer hydrochloride, sevelamer carbonate, or colesevelam hydrochloride, that may be used for treating hyperphosphatemia or reducing cholesterol. The method involves blending of a cross-linked polyallylamine salt with a water soluble excipient, optionally with water, an additive and/or a lubricant, and further tableting the resulting blend to form tablets and tablet cores.

### FIELD OF THE INVENTION

A tablet composition and process for making a composition, tablet or tablet core having cross-linked polyallylamine salts such as sevelamer hydrochloride, sevelamer carbonate, or colesevelam hydrochloride mixed with at least one pharmaceutically acceptable excipient and water.

### BACKGROUND OF THE INVENTION

Polyallylamine hydrochloride salt is produced by polymerization of allylamine hydrochloride in presence of a radical initiator. Polyallylamine hydrochloride is a homopolymer of molecular weight ranging from 2,000 to 100,000 Daltons. Polyallylamine and polyallylamine hydrochloride are highly soluble in water, which upon cross linking with suitable N-alkylating reagents becomes water insoluble, and forms a water swellable polymer.

Cross-linked polyallylamine salts are found to be efficient anion exchange polymers that are insoluble in water. Because of their insolubility in water, cross-linked polyallylamine salts evolved as good phosphate binders and/or bile acid binders without going into a solution form. Thus polyallylamine salts are useful as local acting drug administered to treat hyperphosphatemia and or hyperlipidemia. The protonated polymeric amine groups of cross-linked polyallylamine interact with phosphate ions in the intestine. By binding with phosphate ions in the gastrointestinal tract, the cross-linked polyallylamine reduces absorption of phosphate and lowers phosphate levels in the serum.

Sevelamer hydrochloride (approved by the FDA and sold in the United States under the brand name Renagel®) is a cross-linked polyallylamine hydrochloride having a CH2-CHOH—CH2 group between two amino groups, wherein about 40% of the amine groups are protonated and associated with a chloride anion. Sevelamer hydrochloride contains about 18% chloride by weight, and when administered orally, the chloride anion is exchanged for phosphate anion in the gastrointestinal (GI) system, thus reducing phosphate levels in the blood stream.

Colesevelam hydrochloride (approved by the FDA and sold in the United States under the Brand name Welchol®) is a modified cross-linked polyallylamine polymer containing polyallylamine base moiety having a CH2-CHOH—CH2 group between two amino groups, and is N-alkylated with a specific ratio of (6-trimethylammonium)hexyl and decyl groups. Colesevelam hydrochloride has almost all of its amines protonated and contains about 21% chloride by weight. Colesevelam hydrochloride is a high capacity bile acid binding polymer and is administered orally to treat hyperlipidemia.

Sevelamer carbonate (approved by the FDA sold in the United States under the brand name Renvela®) is yet another cross-linked polyallylamine polymer, having a CH2-CHOH—CH2 group between two amino groups containing bicarbonate anions, and is prescribed to treat hyperphosphatemia. According to the packaging insert of Renvela®, “Sevelamer Carbonate is an anion exchange resin with the same polymeric structure as sevelamer hydrochloride in which carbonate replaces chloride as the counter ion. While the counter ions differ for the two salts, the polymer structure itself, the active moiety, is the same.” The advantage of administering sevelamer carbonate over sevelamer hydrochloride is that in the administration of sevelamer carbonate, the excess load of chloride ions is averted and helps to maintain bicarbonate level in the blood. According to the label and a review of literature for sevelamer carbonate, sevelamer carbonate is made by ion exchange process using sevelamer hydrochloride, by displacing chloride ions with carbonate ions.

The prior art teaches that administration of polyallylamine based polymers are used to treat gout and reduce serum uric acid; to treat Syndrome X; to reduce copper levels and treat copper toxicosis; to reduce levels of fasting plasma glucose and/or hemoglobin A1c; and to reduce LDL-cholesterol. Such polyallylamine based polymer medications are always offered at high doses of about more than 400 mg, and are usually administered orally. The inherent characteristic of these polyallylamine based polymers and/or salts are that these compounds are insoluble and swellable in water, and expand significantly by volume. Because of the water swelling property of polyallylamine based polymers and/or salts, the process of making a reproducible tablet dosage form of these compounds is extremely difficult.

U.S. Pat. No. 7,014,846 (Holmes-Farley et al.) discloses a pharmaceutical composition comprising a carrier and a cross-linked, water insoluble polyallylamine homopolymer, wherein said polyallylamine homopolymer is cross-linked with an epichlorohydrin cross linking agent. One skilled in the art understands that a carrier is a pharmaceutical excipient used for making a pharmaceutical composition wherein the carrier is always present in a level greater than that of the active pharmaceutical ingredient. However, Holmes-Farley does not teach the significance of the carrier or the significance of the level of carrier used in the process of making a composition comprising the carrier and a cross-linked, water insoluble polyallylamine homopolymer. In the examples of Holmer-Farley, a polyallylamine cross-linked with epichlorohydrin ingredient containing about 6% water is disclosed.

U.S. Pat. No. 6,733,780 (Tyler et al.) discloses a tablet comprising a core and a coating thereof, wherein at least about 95% by weight of the core is an aliphatic amine polymer selected from the group consisting of unsubstituted and N-substituted poly(allylamine), poly(diallyl amine), and poly(vinyl amine). Tyler also discloses a tablet wherein the poly(allylamine) comprises from about 3% to about 10% water. Tyler also discloses a tablet comprising a core and a coating therefor, wherein the core comprises 98% by weight sevelamer hydrochloride with a moisture content of 6% by weight, 1% by weight colloidal silicon dioxide and 1% by weight stearic acid, and wherein the coating is a mixture comprising 38.5% w/w low viscosity hydroxypropylmethylcellulose, 38.5% high viscosity hydroxypropylmethylcellulose and 23% w/w diacetylated monoglyceride.

EP Patent No. 0997148 (Matsuda et al.) and U.S. Pat. No. 6,383,518 (Matsuda et al.) disclose tablets which contain phosphate-binding polymers having an average particle size of 400 microns or less, 90% of particles are less than 500 microns and contain crystalline cellulose and/or hydroxypropylcellulose with a low degree of substitution, and the particles also have a moisture content of 1 to 14%. Use of crystalline cellulose as a binder is also claimed. Both Matsuda et al. and Tyler do not disclose a polyol or alcohol based excipients.

WO 01/28527 A2 (Tyler et al.) discloses a tablet core comprising at least about 95% by weight of an aliphatic amine polymer and a process of producing the tablet by hydrating the aliphatic amine polymer to the desired moisture level; blending the aliphatic amine polymer with the excipients in amounts such that the polymer comprises at least about 95% by weight of the resulting blend; and compressing the blend to form tablet core. The tablet is further coated with a water based coating.

PCT Patent Application Publication No. WO 2008/062437 A2 (Hedge et al.) discloses a pharmaceutical composition comprising high shear non-aqueous granulated active ingredient sevelamer hydrochloride in a therapeutically effective amount along with suitable pharmaceutically acceptable excipients. Hedge et al. also discloses that the high shear non-aqueous granulation practiced by this invention improves the cohesiveness of particles and provides excellent flowability and compression characteristics to the tablet. One of the drawbacks of the composition disclosed in Hedge et al. is that use of non aqueous solvent in the granulation causes severe drying problems and always results in high levels of residual solvent.

PCT Patent Application Publication No. WO 2007/094779 A1 (Hrakovsky et al.) and US Patent Application Publication No. US 2007/190020 A1 (Hrakovsky et al.) disclose a pharmaceutical composition comprising: a) wet granulated aliphatic amine polymers; and b) at least one pharmaceutical excipient. Also disclosed in Hrakovsky is a method of making tablet using granulating solution, wherein the granulating solution is an ethanol/water solution prepared from about 82% to about 95% of Ethanol (95%) and about 5% to about 18% water. Though the granulation liquid contains less than 18% water, the major component of the solution (ethanol or alcohol) would result in very high levels of residual alcohol in the product which is not acceptable for high dose products such as sevelamer hydrochloride and sevelamer carbonate tablets.

U.S. Pat. No. 6,756,364 (Barbier et al.) discloses a pharmaceutical composition comprising a lipase inhibitor and a pharmaceutically acceptable bile acid sequestrant, wherein the pharmaceutically acceptable bile acid sequestrant is selected from the group consisting of cholestyramine, Colestipol, colesevelam, cholestimide, sevelamer, cellulose and dextran derivatives, starch and starch derivatives and pharmaceutically acceptable salts thereof. The binders used are a cellulose or a dextran derivative. The dextran derivative is selected from the group consisting of DEAE-cellulose, guanidinoethylcellulose, and DEAE-Sephadex. The starch derivative is selected from the group consisting of P- or y-cyclodextrin, retrograded and/or degraded starch, hydrophobic starch, amylose, starch diethylaminoethyl ether and starch-2-hydroxyethylether.

Canadian Patent No. CA 2387915 A1 (Tyler et al.) discloses a tablet comprising a core and coating therefor, wherein at least about 95% by weight of the core is an aliphatic amine polymer selected from the group consisting of unsubstituted and N-substituted poly(allylamine), poly(diallylamine) and poly(vinyl amine). The polyallylamine used in the tablet is hydrated and contains about 3% to 10% water. Also used in the composition are hydroxypropylmethylcellulose and a plasticizer.

Sevelamer hydrochloride and sevelamer carbonate hydrochloride tablets disclosed in the prior art require the active phosphate binding ingredient to have a water content of about 1 to 14% to form a tablet or tablet core. The phosphate binding agent is chemically derived from polyallylamine cross-linked with epichlorohydrin and exhibits high swelling ability upon contact with water. Such a moisture treated or moisture containing active ingredient is one of the key elements of the prior art process of making tablets.

An inherent property of cross-linked polyallylamine based compounds is swelling through the absorption of water and/or solvent. Swelling is a reversible process and the material that swelled always reverts to its original state upon removing the absorbed solvent by evaporation. Therefore the prior art requirement of having about 6% water content in the wet cross-linked polyallylamine compounds is very difficult to maintain throughout the shelf life of the drug substance. Moreover controlling particle size of the wet swelled drug substance is very difficult and rarely reproducible and always adds uncertainties to the process parameters. Colesevelam hydrochloride (approved by the FDA and sold in the United States under the Brand name Welchol®) tablet contains micro crystalline cellulose as one of its excipients. Microcrystalline cellulose is not soluble in water and thus when administered orally it remains as an insoluble particle in the gastrointestinal tract. U.S. Pat. No. 5,091,192 (See) has disclosed that insoluble cellulose binds permanently with bile acids. Therefore cellulose itself being a bile acid binder, it would certainly interfere with the bile acid binding capacity of colesevelam hydrochloride, and this interference would cause severe improper dose assessment. To have a perfect oral dosage form of colesevelam hydrochloride with desired dosage strength, it is necessary to have excipients that do not bind with bile acids.

To solve the problems in the prior art, the present invention discloses a composition and process for making an oral dosage form such as a tablet, caplet, capsule, powder, as well as other dosage forms understood in the art, by employing either substantially dry or hydrated cross-linked polyallylamine based drug substances. This invention discloses the use of polyhydroxy compounds as binder/filler to form a direct compression tablet of cross-linked polyallylamine salts that are substantially dry or significantly wet, and also a process of making the same.

Additionally, the invention discloses the use of water based excipients that are polyols and to add these polyols to cross-linked polyallylamine salts in order to form substantially dry or hydrated cross-linked polyallylamine based drug substances. These formulations are useful to treat hyperphosphatemia and/or reduce cholesterol, and have advantageous tableting advantages.

### SUMMARY OF THE INVENTION

The present invention provides a composition of matter for an oral dosage form containing at least one substantially dry or hydrated cross-linked polyallylamine based active ingredient useful to treat hyperphosphatemia and/or reduce cholesterol; one or more excipients selected from the group consisting of a water soluble filler material, such as sorbitol, xylitol, maltitol, sucrose, dextrose, dextrate, maltose, Isomalt, maltodextrin, lactitol, monosaccharide, disaccharide, polyethylene glycol etc; and optional water and optional lubricants and other excipients known in use with polyallylamine based active ingredients. The oral dosage form may consist of a transmucosal lozenge, sublingual tablet, oral tablet, rapidly disintegrating tablet, caplet, hard capsule, soft capsule, cachet, troche, or dissolvable tab, as well as other dosage forms known in the art.

In a preferred embodiment, the pharmaceutical composition comprises at least one cross-linked polyallylamine salt, and at least one water soluble excipient that is a polyol.

The composition may further comprise at least one lubricant, at least one additive, and/or water. The cross-linked polyallylamine salt may be sevelamer hydrochloride, sevelamer carbonate, or colesevelam hydrochloride. The lubricant may be selected from a group consisting of stearic acid, sodium stearate, magnesium stearate, calcium stearate, zinc stearate, or sodium stearylfumarate, or a combination thereof. The additive may be selected from a group consisting of silicon dioxide, sodium chloride, or sodium carbonate, or a combination thereof.

The composition may be milled into a tablet or tablet core. The amount of cross-linked polyallylamine salt may be about 60% to 90% by weight. The amount of the at least one excipient is about 5% to 35% by weight. The at least one excipient may be selected from a group consisting of sorbitol, xylitol, maltitol, Mannitol, dextrose, sucrose, dextrate, Isomalt, maltose, lactitol, maltodextrin, polyethylene glycol, monosaccharide, or disaccharide.

The cross-linked polyallylamine salt may also have a water content of less than 3%. The particle size of the cross-linked polyallylamine salt may be less than 400 microns, more preferably to less than 250 microns, and most preferably to less than 150 microns.

The present invention also discloses a method and process for making such tablets and/or tablet cores using a substantially dry or significantly wet cross-linked polyallylamine salt such as sevelamer hydrochloride, sevelamer carbonate, or colesevelam hydrochloride, or a combination of these salts, and combining this salt or salt mixture with a pharmaceutically acceptable binder and optionally with water. One specific embodiment disclosed in this invention provides a composition of a pharmaceutical solid dosage form comprising sevelamer hydrochloride or sevelamer carbonate or colesevelam hydrochloride with a binder selected from the group consisting of water soluble filler material, such as sorbitol, xylitol, maltitol, sucrose, dextrose, dextrate, maltose, Isomalt, maltodextrin, lactitol, monosaccharide, disaccharide, polyethylene glycol etc, optional water and optional lubricant, or a combination thereof.

A preferred embodiment involves a method of making a tablet blend composition comprising at least one cross-linked polyallylamine salt useful to treat hyperphosphatemia and or reducing cholesterol, comprising the steps: (a) milling the substantially dry cross-linked polyallylamine salt to a desired particle size, (b) blending with at least one water soluble excipient, and (c) optionally blending with water after the step (a) or (b), (d) optionally blending the mixture obtained in step (c) with an additive and or lubricant.

The method may further comprise a step of reducing the particle size of said cross-linked polyallylamine to less than 400 microns, wherein said reducing step occurs before step (a). More preferably the step step will reduce the particle size of the cross-linked polyallylamine salt to less than 250 microns, and most preferably to less than 150 microns

The method may further comprise adding water to the cross-linked polyallylamine salt. The method may further involve coating said tablet or said tablet core. The method may further have at least one water soluble excipient selected from the group consisting of sorbitol, xylitol, maltitol, Mannitol, dextrose, sucrose, dextrate, polyethylene glycol, monosaccharide, or disaccharide, or a combination thereof.

The method may further comprise blending the mixture of step (a) or step (b) with water. The method may further comprise blending the mixture obtained in step (c) with an additive or lubricant, or both.

In a preferred embodiment, the cross-linked polyallylamine salt used in the method has a water content of less than 3%.

An embodiment of the invention involves a method of making a composition comprising the steps of: (1) mixing at least one substantially dry or hydrated cross-linked polyallylamine salt with at least one excipient; (2) blending the mixture obtained in step 1 optionally with water; (3) tableting the blend obtained in step 2.

The excipient may be selected from the group consisting of water soluble filler material, such as sorbitol, xylitol, maltitol, sucrose, dextrose, dextrate, maltose, Isomalt, maltodextrin, lactitol, stevia (natural sweetener), monosaccharide, disaccharide, polyethylene glycol, water or a combination thereof.

The cross-linked polyallylamine salt may be selected from the group consisting of sevelamer, sevelamer hydrochloride, sevelamer carbonate, or colesevelam hydrochloride, or a combination thereof.

The method may further comprise adding a lubricant to step (3) of the method. The lubricant may be selected from the group consisting of stearic acid, sodium stearate, magnesium stearate, sodium stearylfumarate, a metal salt, or a combination thereof. The metal salt may be selected from the group consisting of a salt of sodium, magnesium, or calcium, or a combination thereof.

The method may involve no drying step and tableting the composition using a tablet press. External lubricating agents may also be used while tableting.

Another embodiment of the invention involves a method of making a composition comprising of mixing at least one substantially dry cross-linked polyallylamine salt with at least one excipient, and water as part of the product.

The composition of the present invention may be useful to treat hyperphosphatemia or reduce cholesterol. The excipient in the composition may be selected from the group consisting of water soluble filler material, such as sorbitol, xylitol, maltitol, sucrose, dextrose, dextrate, maltose, Isomalt, maltodextrin, lactitol, monosaccharide, disaccharide, polyethylene glycol or a combination thereof.

The cross-linked polyallylamine salt may be selected from the group consisting of sevelamer hydrochloride, sevelamer carbonate, sevelamer bicarbonate, or colesevelam hydrochloride, or a combination thereof.

The method may further comprise adding a lubricant to the step of mixing at least one substantially dry or hydrated cross-linked polyallylamine salt with at least one water soluble excipient, and water. The lubricant may be selected from the group consisting of stearic acid, sodium stearate, magnesium stearate, sodium stearylfumarate, or a metal salt. The metal salt may be selected from the group consisting of a salt of sodium, magnesium, calcium, or a combination thereof.

Another embodiment of the invention involves a composition comprising at least one substantially dry cross-linked polyallylamine salt, at least one excipient, and water.

The at least one excipient may be selected from the group consisting of water soluble filler material, such as sorbitol, xylitol, maltitol, sucrose, dextrose, dextrate, maltose, Isomalt, maltodextrin, lactitol, monosaccharide, disaccharide, polyethylene glycol etc, or a combination thereof.

Optionally the composition disclosed in this invention contains an additive selected from the group of water soluble acid addition salt of a base. The acid addition salts are sodium chloride or sodium carbonate or ammonium chloride, tertraalkylammonium halides. One composition disclosed in this invention contains silicon dioxide as additive.

The composition may be administered to a human being and may be administered to treat hyperphosphatemia or reduce cholesterol. Other diseases known in the art may also be treated with the composition.

The cross-linked polyallylamine salt in the tablet may be selected from the group consisting of sevelamer hydrochloride, sevelamer carbonate, or colesevelam hydrochloride, or a combination thereof.

The composition may further comprise a lubricant. The lubricant may be selected from the group consisting of stearic acid, sodium stearate, magnesium stearate, sodium stearylfumarate, or a metal salt.

The metal salt may be selected from the group consisting of sodium, magnesium, calcium, or a combination thereof.

The composition may be administered in a dosage form selected from the group consisting of a transmucosal lozenge, sublingual tablet, oral tablet, rapidly disintegrating tablet, caplet, hard capsule, soft capsule, cachet, troche, or dissolvable tab, or combination thereof.

The invention also refers to a kit to treat a blood disorder comprising (a) a composition of at least one substantially dry cross-linked polyallylamine salt, at least one water soluble excipient, and water, and (b) a label indicating administration in a dosage form.

The kit may be used to treat hyperphosphatemia and/or reduce cholesterol. The dosage form revealed by the kit may be selected from the group consisting of a transmucosal lozenge, sublingual tablet, oral tablet, rapidly disintegrating tablet, caplet, hard capsule, soft capsule, cachet, troche, or dissolvable tab, or combination thereof.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### DETAILED DESCRIPTION OF INVENTION

Polyallylamine based phosphate and or bile acid binders have swelling characteristics that differ with the amount of solvent the binder absorbs. Making tablets with cross-linked polyallylamine based active ingredients containing from about 1% water to 14% water, with common dry excipients such as binders, fillers and other such excipients known in the art used for tableting, is very challenging while employing a large variation in water content of the active ingredient (1 to 14%). This requires a large variation in the filler/binder excipient quantity sufficient to maintain the tablet weight.

The practical effects of adjusting an excipient which is less than 10% weight of a tablet of cross-linked polyallylamine salt containing a wide range of water content is provided in Table-1. For example for a given tablet weight of 800 mg strength, containing a cross-linked polyallylamine salt with 14% water, having its excipient at 5% of the tablet weight, requires about 48 mg of excipient to provide a total tablet weight of 960 mg. To make the same size tablet with a drug containing less than 1% water, the required amount of excipient is about 160 mg. This is about 4 times the quantity of excipient required for the tablet made with 14% water. Where there is such a large variation in excipient quantity to maintain the tablet weight because the drug substance has a variable water content, requires special attention to the choice of excipient selected. Variation in excipient quantity by 4 times would greatly affect the tablet characteristics if the excipient chosen has significant interaction with the drug upon contact with water. None of the prior art has disclosed such difficulties in making tablets with cross-linked polyallylamine salts.

The present invention provides a novel solution to the above problem by disclosing a novel composition wherein the cross-linked polyallylamine salt has at least interaction with the excipient upon contact with water. The excipients disclosed in this invention suitable to make tablets of cross-linked polyallylamine salts are water soluble excipients such as poly hydroxy compounds. In addition to using a binder, such poly hydroxy compounds may also use an additive such as sodium chloride, sodium carbonate, silicon dioxide alone or in combination to help to improve the disintegration of the tablet. Use of disintegrants such as croscarmellose sodium, povidone and sodium starch glycolate are found to exhibit improper disintegration and form cloudy lumps.

TABLE 1
Composition of a tablet containing 800 mg drug containing 1 to
14% water and its impact on the level of excipient

| | Drug wt | | Drug as is, | | | |
|---|---|---|---|---|---|---|
| | (anhydrous | Water | (Drug + water) | Excipient | | Tablet |
| # | basis), mg | in the drug % | wt, mg | mg | Excipient % | wt. mg |
| 1 | 800 | 0 | 800 | 160.0 | 16.7 | 960.0 |
| 2 | 800 | 1 | 808 | 152.0 | 15.8 | 960.0 |
| 3 | 800 | 5 | 840 | 120.0 | 12.5 | 960.0 |
| 4 | 800 | 7 | 856 | 104.0 | 10.8 | 960.0 |
| 5 | 800 | 10 | 880 | 80.0 | 8.3 | 960.0 |
| 6 | 800 | 12 | 896 | 64.0 | 6.7 | 960.0 |
| 7 | 800 | 14 | 912 | 48.0 | 5.0 | 960.0 |

Most of the commonly used excipients do not fulfill this requirement and would not form a direct compression tablet of required hardness and disintegration characteristics. Attempts to make tablets with sevelamer hydrochloride containing water at a level of about 3% and of average particle size about 250 to 500 micron and excipients such as lactose or starch are relatively soft and break down easily. These tablets are not suitable for manufacturing. Also tablets made with commonly used disintegrants such as croscarmellose sodium, sodium starch glycolate or povidone are found to show poor disintegration characteristics than the tablet made without a disintegrant. These disintegrants are ionic in nature and tend to swell in water exhibiting the disintegration property.

According to one embodiment of this invention it is found that use of disintegrants to make tablets of cross-linked polyallylamine salts inhibit the tablet from disintegration by forming ionic cloud around the tablet surface due to strong ionic interaction between the cross-linked polyallylamine salt and the disintegrant. Use of povidone has the worst effect as it holds the drug particles together by ionic cluster interaction and does not allow the tablet to disintegrate quickly. Thus use of such ionic excipients such as sevelamer hydrochloride, sevelamer carbonate, colesevelam hydrochloride, upon contact with water form a cluster around the ionic polymer drug particle and thus inhibit the disintegration of the particles in aqueous media. A solution to overcome this ionic interaction between the drug, cross-linked polyallylamine drug substance and the excipients has been disclosed in this invention by offering water soluble polyhydroxy compounds to form the tablet. Upon contact with water such water soluble excipients dissolve in water and release the cross-linked polyallylamine salt drug substances which are self disintegrating.

The polyhydroxy compounds found suitable for making tablets of cross-linked polyallylamine salts are sorbitol, xylitol, maltitol, Mannitol, dextrose, sucrose, dextrate, Isomalt, maltose, lactitol, maltodextrin, polyethylene glycol, monosaccharide, and disaccharide. An alternate approach is a wet granulation technique employing mixture of organic solvent and water, which provides a process of making good tablets but the tablets contain higher level of organic residual solvent that is not removable by drying and hence not acceptable for administration.

Controlling particle size as well as the content of water in the active ingredient are two important parameters in making a solid dosage form. Especially with water swellable polymeric active ingredients, controlling particle size and maintaining specified water content is difficult and always leads to quality problems. Making tablets by directly compressing such large dose swellable polymers with common excipients results in soft tablets that cannot meet the set minimum quality standards for a tablet. Most of the excipients that are suitable for making tablets with wet sevelamer hydrochloride are not suitable for making tablets with dry sevelamer hydrochloride. In order to circumvent this issue most of the prior art have used sevelamer hydrochloride containing about 6% water to form tablets with acceptable physical properties.

Controlling particle size of hydrated cross-linked polyallylamine salts is generally more difficult than controlling particle size of a substantially dry material. The hydrated material upon milling causes lump formation. As such, controlling particle size reduction of hydrated cross-linked polyallylamine salts having a water content from about 6 to about 14% is difficult to achieve in large scale operation, where as size reduction of the dried cross-linked polyallylamine salts having low water content from about 0.1% to 3% is much more straight forward and easier for scale up operations. Substantially dry cross-linked polyallylamine salts may be milled in a Fitz mill/Fitz sieve in presence of nitrogen to avoid further moisture pickup. The particle size distribution of the milled cross-linked polyallylamine salt may be less than about 400 μm, preferably less than 250 μm, more preferably less than 100 μm, and most preferably less than 75 μm. Preparation of a direct compression tablet dosage form using a substantially dried cross-linked polyallylamine salt may be made by combining the salt with suitable excipients that are water soluble polyhydroxy compounds.

The advantage of using a dried cross-linked polyallylamine salt is (1) reproducing consistent particle size control between batches, (2) achieving the desired water content in the blend by adding additional water.

A novel composition and process of making substantially hard tablets of cross-linked polyallylamine salt such as sevelamer hydrochloride, sevelamer carbonate, and colesevelam hydrochloride can be made by the disclosure of invention, using certain excipients and optionally using additional water in the blending process. An embodiment of the present invention discloses a process of making a tablet or tablet core using a cross-linked polyallylamine salt such as sevelamer hydrochloride, sevelamer carbonate, colesevelam hydrochloride, with a pharmaceutically acceptable water soluble filler, and optionally adding a lubricant and/or water.

Also disclosed in this invention is that the tablets made with sevelamer hydrochloride, sorbitol, an optionally an additive and/or lubricant, such as stearate salts, are stronger and yield harder tablets. In one of the examples of this invention, sevelamer hydrochloride containing about 0.5% moisture is blended with sorbitol and made into tablet without any sticking or chipping or ejection problems, and the tablet is smooth, excellently hard and disintegrates in dissolution medium in a time period of less than 10 minutes. Such a tableting process is easily adaptable to any manufacturing scale without any difficulties. In another example of the invention, use of wet sevelamer hydrochloride (of moisture about 10%) was mixed with sorbitol and punched in to tablets of same weight as that of the tablets made with substantially dry sevelamer hydrochloride (containing about 0.5% moisture). The tablets made with sorbitol and dry or hydrated sevelamer hydrochloride are physically similar in characteristics, and exhibit excellent tablet properties and excellent tableting process characteristics such as very minimum ejection pressure, consistent formation when punched (no broken tablets from the punching process), hardness, smoothness, and other desired tableting process characteristics.

The process of this invention involves using water in the blend of polyallylamine based active ingredient and at least one of any polyol filler and or a wet disintegrant to make the tablet making process reliable and consistently reproducible. By using external water in the blend, it helps to select an excipient that is generally water soluble. Excipients such as disintegrants characteristically absorb water and exhibit swelling. Though a disintegrant usually helps to retain the water, use of such disintegrant in sevelamer hydrochloride tablet formation helps to hold the polymer particles together in the tablet, but shows poor disintegration in a water medium. Also use of additional water to form the tablet may be very critical as the water added causes significant expansion of the blend volume and causes tablet weight adjustment problems. The ionic nature of a disintegrant such as croscarmellose sodium, sodium starch glycolate, povidone, and others, forms a cloudy layer around the tablet, the tablet is inhibited from disintegration of the sevelamer hydrochloride particles. However, tablets made with sevelamer hydrochloride and sorbitol without any additional disintegrants are found to show excellent hardness as well uniform disintegration. The cross-linked polyallylamine salt itself has swelling characteristics, the use of water soluble binder helps to bind the drug particles together in the tablet, and the particles in the tablet are released in aqueous medium upon dissolution of the excipient. According to the present invention, excipients suitable to make a cross-linked polyallylamine salt tablet soluble in water and their solubilities are listed in Table-2.

TABLE 2
Solubility of water soluble excipients:

| | | Solubility | Reference: |
|---|---|---|---|
| | Polyhydroxy | at 20° C., in | Handbook of Pharmaceutical |
| # | compounds | water | Excipients 5^{th} Edn. |
| 1 | Sorbitol | 1 in 0.5 | Page No. 719 |
| 2 | Maltitol | 1 in 0.67 | Page No. 439 |
| 3 | Xylitol | 1 in 1.6 | Page No. 825 |
| 4 | Mannitol | 1 in 5.5 | Page No. 451 |
| 5 | Sucrose | 1 in 0.5 | Page No. 745 |
| 6 | Dextrose | 1 in 1 | Page No. 232 |
| | (monohydrate) | | |
| 7 | Dextrate | 1 in 1 | Page No. 226 |
| 8 | Isomalt | 1 in 2.5 | Page No. 368 |
| 9 | Maltose | 1 in less than 1 | Page No. 448 |
| 10 | Polyethylene glycol | 1 in 10 to 30 | Page No. 546 |
| | (types from 1,000 | | |
| | to 20,000) | | |
| 11 | Lactitol | 1 in 1.75 | Page No. 384 |
| 12 | Maltodextrin | 1 in 1 to 10 | Page No. 443 |

Specific embodiments as disclosed in this invention provide a composition of a pharmaceutical solid dosage form comprising a cross-linked polyallylamine salt, a filler selected from one or more excipients selected from the group sorbitol, xylitol, maltitol, mannitol, dextrose, dextrate, polyethylene glycol, sucrose, monosaccharide, disaccharide and optionally a lubricant and/or water.

According to one embodiment of this invention, disclosed is a pharmaceutical tablet or tablet core composition comprising at least one of the cross-linked polyallylamine based ingredient selected from the group sevelamer hydrochloride, sevelamer carbonate and colesevelam hydrochloride, a polyol and optionally an additive and/or lubricant. The polyol disclosed in this invention are selected from a group of sorbitol, maltitol, xylitol, mannitol, dextrose, dextrate, polyethylene glycol, sucrose or similar polyhydroxy compounds. The additive is sodium chloride, sodium carbonate, or silicon dioxide. The sum of cross-linked polyallylamine salt and water is less than about 90% of the blend weight.

According to one embodiment, disclosed is a process of making a sevelamer hydrochloride tablet or tablet core comprising:
1. milling substantially dry sevelamer hydrochloride to reduce particle size;
2. blending the milled material with at least one polyol selected from the group sorbitol, maltitol, xylitol, sucrose, maltose, dextrose, dextrate, polyethylene glycol;
3. optionally blending the mixture obtained in step 2 with an additive and/or a lubricant; and
4. making tablets by direct compression using a tablet press.

Optionally the milled dry sevelamer hydrochloride is hydrated with addition of water to about from 2% to 8% either before or after step (2) above. The use of water soluble polyhydroxy compounds helps to distribute the water uniformly throughout the matrix.

The lubricants used are sodium stearate, magnesium stearate, calcium stearate, sodium stearylfumarate among other such lubricants known in the art. The tablets made by the process of this invention are excellently hard and shows excellent disintegration properties. These tablets may be coated with suitable pharmaceutically acceptable coating mixtures.

The additives used are sodium chloride, sodium carbonate, silicon dioxide or combination thereof.

A similar procedure may be also employed to prepare a tablet core of sevelamer carbonate or colesevelam hydrochloride. The tablets prepared according to the process of this invention are tested for disintegration time in water at 37° C.

### EXAMPLES

### 1. Preparation of Sevelamer Hydrochloride Tablet

About 42.5 g sevelamer hydrochloride (containing a moisture level of about 6% and particle size less than 250 μm) was mixed with 8 g of croscarmellose sodium and blended for about 5 minutes. To this mixture, about 1.5 g of water was added and blended well. About 200 mg sodium stearate was added and blended for 3 minutes. Tablets were made by transferring about 522 mg blend into the dye and punched at a pressure of about 3 tons.

Composition of the tablet core:
Sevelamer hydrochloride: 400 mg
(Water from sevelamer hydrochloride: 25 mg)
Croscarmellose sodium: 80 mg

### Added Water: 15 mg

### Sodium Stearate: 2 mg

Disintegration time in water at 37° C.: 15 minutes

### 2. Preparation of Sevelamer Carbonate Tablet

About 42.5 g sevelamer carbonate (containing a moisture level of about 6% and particle size less than 250 μm) was mixed with 8 g of croscarmellose sodium and blended for about 5 minutes. About 200 mg sodium stearate was added and blended for 3 minutes. Tablets were made by transferring about 507 mg blend into the dye and punched at a pressure of about 3 tons.

Composition of the tablet core:
Sevelamer carbonate: 400 mg
(Water from Sevelamer carbonate: 25 mg)
Croscarmellose sodium: 80 mg
Sodium stearate: 2 mg
Disintegration time in water at 37° C.: 15 minutes

### 3. Preparation of Sevelamer Hydrochloride Tablet

About 40.3 g dry Sevelamer hydrochloride (containing moisture less than 0.5% and particle size less than 100 μm) was mixed with 8 g of povidone and blended for about 5 minutes. About 200 mg sodium stearate was added and blended for 3 minutes. Tablets were made by transferring about 482 mg of the blend into the dye and punched at a pressure of about 3 tons.

Composition of the tablet core:
Sevelamer hydrochloride: 400 mg
(Water from sevelamer hydrochloride: 3 mg)

### Povidone: 77 mg

Sodium stearate: 2 mg
Disintegration time in water at 37° C.: 30 minutes

### 4. Preparation of Sevelamer Hydrochloride Tablet

About 42.5 g Sevelamer hydrochloride (containing moisture of about 6% and particle size less than 100 μm) was mixed with 8 g of polyethylene glycol 8000 and blended for about 5 minutes. About 200 mg sodium stearate was added and blended for 3 minutes. Tablets were made by transferring about 507 mg blend into the dye and punched at a pressure of about 3 tons.

Composition of the tablet core:
Sevelamer hydrochloride: 400 mg
(Water from sevelamer hydrochloride: 25 mg)
polyethylene glycol 8000: 80 mg

### Sodium Stearate: 2 mg

Disintegration time in water at 37° C.: 5 minutes

### 5. Preparation of Sevelamer Hydrochloride Tablet Using Substantially Hydrated Sevelamer Hydrochloride

About 44 g Sevelamer hydrochloride (containing moisture of about 10% and particle size less than 75 μm) was mixed with 10 g of spray dried sorbitol (Roquette Neosorb P60W) and blended for about 5 minutes. About 200 mg sodium stearate was added and blended for 2 minutes. Tablets were made by transferring about 542 mg blend into the dye and punched at a pressure of about 3 tons.

Composition of the tablet core:
Sevelamer hydrochloride: 400 mg
(Water from sevelamer hydrochloride: 40 mg)

### Sorbitol: 100 mg

Sodium stearate: 2 mg
Disintegration time in water at 37° C.: 4 minutes

Also tablets were made with 1084 mg of the blend to form a tablet equivalent to 800 mg sevelamer hydrochloride.

Composition of the tablet core:
Sevelamer hydrochloride: 800 mg
(Water from sevelamer hydrochloride: 80 mg)

### Sorbitol: 200 mg

Sodium stearate: 4 mg
Disintegration time in water at 37° C.: 9 minutes

### 6. Preparation of Sevelamer Carbonate Tablet

About 40.2 g sevelamer carbonate (containing moisture of about 0.4% and particle size less than 100 μm) was mixed with 2.0 g of silicon dioxide (SIPERNAT) and 12.5 g of spray dried sorbitol (Roquette Neosorb P60W) and blended for about 5 minutes. About 200 mg sodium stearate was added and blended for 2 minutes. Tablets were made by transferring about 549 mg blend into the dye and punched at a pressure of about 3 tons.

Composition of the tablet core:

### Sevelamer Carbonate: 400 mg

(Water from sevelamer carbonate: 2 mg)

### Sorbitol: 125 mg

Silicon dioxide: 20 mg
Disintegration time in water at 37° C.: 3 minutes

### 7. Preparation of Sevelamer Carbonate Tablet

About 40.2 g dry sevelamer carbonate (containing moisture of about 0.4% and particle size less than 100 μm) was mixed with 2.0 g of silicon dioxide (SIPERNAT) and 12.5 g of spray dried sorbitol (Roquette Neosorb P60W) and blended for about 5 minutes. About 100 mg sodium stearate was added and blended for 2 minutes. Tablets were made by transferring about 549 mg blend into the dye and punched at a pressure of about 3 tons.

Composition of the tablet core:

### Sevelamer Carbonate: 400 mg

(Water from sevelamer carbonate: 2 mg)

### Sorbitol: 127 mg

Silicon dioxide: 10 mg
Sodium stearate: 8 mg
Disintegration time in water at 37° C.: 4 minutes

### 8. Preparation of Sevelamer Hydrochloride Tablet Using Substantially Dry Sevelamer Hydrochloride

About 40.3 g dry sevelamer hydrochloride (containing moisture of about 0.5% and particle size less than 100 μm) was mixed with 13.7 g of spray dried sorbitol (Roquette Neosorb P60W) and blended for about 5 minutes. About 200 mg sodium stearate was added and blended for 2 minutes. Tablets were made by transferring about 542 mg blend into the dye and punched at a pressure of about 3 tons.

Composition of the tablet core:

### Sevelamer Hydrochloride: 400 mg

(Water from sevelamer Hydrochloride: 3 mg)

### Sorbitol: 137 mg

Sodium stearate: 2 mg
Disintegration time in water at 37° C.: 4 minutes

Also tablets were made with 1084 mg of the blend to form a tablet equivalent to 800 mg sevelamer hydrochloride.

Composition of the tablet core:

### Sevelamer Hydrochloride: 800 mg

(Water from sevelamer Hydrochloride: 6 mg)

### Sorbitol: 274 mg

Sodium stearate: 4 mg
Disintegration time in water at 37° C.: 8 minutes

### 9. Preparation of Sevelamer Hydrochloride Tablet Using Additive

About 40.3 g dry sevelamer hydrochloride (containing moisture of about 0.5% and particle size less than 100 μm) was mixed with 1.0 g of silicon dioxide (SIPERNAT) and 13.7 g of spray dried sorbitol (Roquette Neosorb P60W) and blended for about 5 minutes. About 400 mg sodium stearate was added and blended for 2 minutes. Tablets were made by transferring about 554 mg blend into the dye and punched at a pressure of about 3 tons.

Composition of the tablet core:

### Sevelamer Hydrochloride: 400 mg

(Water from sevelamer Hydrochloride: 3 mg)
Silicon dioxide: 10 mg

### Sorbitol: 137 mg

Sodium stearate: 4 mg
Disintegration time in water at 37° C.: 1.4 minutes

Also tablets were made with 1108 mg of the blend to form a tablet equivalent to 800 mg sevelamer hydrochloride.

Composition of the tablet core:

### Sevelamer Hydrochloride: 800 mg

(Water from sevelamer Hydrochloride: 6 mg)
Silicon dioxide: 20 mg

### Sorbitol: 274 mg

Sodium stearate: 8 mg
Disintegration time in water at 37° C.: 3 minutes

### 10. Preparation of Colesevelam Hydrochloride Tablet

About 41.6 g colesevelam hydrochloride (containing moisture about 4% and particle size less than 75 μm) was mixed with 1.0 g silicon dioxide, 10 g of sorbitol and blended for about 5 minutes. About 200 mg of magnesium stearate was added and blended for about 2 minutes. Tablets were made by transferring about 528 mg blend into the dye and punched at a pressure of about 3 tons.

Composition of the tablet core:
Colesevelam hydrochlorid: 400 mg
(Water from Colesevelam Hydrochloride: 16 mg)
Silicon dioxide: 10 mg

### Sorbitol: 100 mg

Magnesium stearate: 2 mg
Disintegration time in water at 37° C.: 4 minutes

### 11. Preparation of Sevelamer Hydrochloride Tablet

About 41.2 g sevelamer hydrochloride (containing moisture of about 3% and particle size less than 100 μm) was mixed with 1 g of sodium chloride powder and 12.5 g of sugar 6× and blended for about 5 minutes. About 200 mg sodium stearate was added and blended for 2 minutes. Tablets were made by transferring about 549 mg blend into the dye and punched at a pressure of about 3 tons.

Composition of the tablet core:

### Sevelamer Hydrochloride: 400 mg

(Water from sevelamer Hydrochloride: 12 mg)

### Sugar 6×: 125 mg

Sodium chloride: 10 mg
Sodium stearate: 2 mg
Disintegration time in water at 37° C.: 3 minutes

### 12. Preparation of Sevelamer Hydrochloride Tablet

About 41.2 g sevelamer hydrochloride (containing moisture of about 3% and particle size less than 100 μm) was mixed with 1.0 g sodium carbonate and 12.5 g of Mannitol (Pearlitol) and blended for about 5 minutes. About 200 mg sodium stearate was added and blended for 2 minutes. Tablets were made by transferring about 549 mg blend into the dye and punched at a pressure of about 3 tons.

Composition of the tablet core:
Sevelamer hydrochloride: 400 mg
(Water from sevelamer Hydrochloride: 12 mg)
Sodium carbonate: 10 mg

### Mannitol: 125 mg

Sodium stearate: 2 mg
Disintegration time in water at 37° C.: 5 minutes

### 13. Preparation of Sevelamer Hydrochloride Tablet by Addition of Water

About 41.2 g sevelamer hydrochloride (containing moisture of about 3%) was milled to reduce the particle size to less than 75 μm, and transferred to a granulation bowl, where 2.0 g water was added slowly under stirring for about 15 min, and further mixed with 12.5 g of mannitol and blended for about 10 minutes. About 200 mg sodium stearate was added and blended for 2 minutes. Tablets were made by transferring about 559 mg blend into the dye and punched at a pressure of about 3 tons.

Composition of the tablet core:
Sevelamer hydrochloride: 400 mg
(Water from sevelamer Hydrochloride: 12 mg)

### Mannitol: 125 mg

### Added Water: 20 mg

Sodium stearate: 2 mg
Disintegration time in water at 37° C.: 8 minutes

### 14. Preparation of Sevelamer Hydrochloride Tablet by Addition of Water

About 41.2 g sevelamer hydrochloride (containing moisture of about 3%) was milled to reduce the particle size to less than 75 μm, and transferred to a granulation bowl, 2.0 g water was added slowly under stirring for about 15 min, and further mixed with 12.5 g of mannitol and blended for about 10 minutes. About 2.0 g sodium stearate was added and blended for 2 minutes. Tablets were made by transferring about 577 mg blend into the dye and punched at a pressure of about 3 tons.

Composition of the tablet core:

### Sevelamer Hydrochloride: 400 mg

(Water from sevelamer Hydrochloride: 12 mg)

### Mannitol: 125 mg

### Added Water: 20 mg

Sodium stearat: 20 mg
Disintegration time in water at 37° C.: 8 minutes

### 15. Preparation of Sevelamer Hydrochloride Tablet by Addition of Water

About 41.2 g sevelamer hydrochloride (containing moisture of about 3%) was milled to reduce the particle size to less than 75 μm, and transferred to a granulation bowl, blended with 12.5 g of Sorbitol for about 10 minutes, and further blended by addition of 2.5 g water slowly under stirring for about 15 min, and blended for about 10 additional minutes. About 400 mg sodium stearate was added and blended for 2 minutes. Tablets were made by transferring about 551 mg blend into the dye and punched at a pressure of about 3 tons.

Composition of the tablet core:
Sevelamer hydrochloride: 400 mg
(Water from sevelamer Hydrochloride: 12 mg)

### Sorbitol: 125 mg

### Added Water: 25 mg

Sodium stearate: 4 mg
Disintegration time in water at 37° C.: 4 minutes

While the invention has been specifically described in connection with certain specific embodiments thereof, it is to be understood that this is by way of illustration and not of limitation and that various changes and modifications in form and details can be made thereto, and the scope of the appended claims should be construed as broadly as the prior art will permit.

The description of the invention is merely exemplary in nature, and thus, variations that do not depart from the gist of the invention are intended to be within the scope of the invention. Such variations are not to be regarded as a departure from the spirit and scope of the invention.

What is claimed is:

1. A pharmaceutical composition useful to treat hyperphosphatemia or reduce cholesterol comprising:
at least one cross-linked polyallylamine salt, and at least one water soluble excipient that is a polyol.

2. A method of making a tablet blend composition comprising at least one cross-linked polyallylamine salt useful to treat hyperphosphatemia and or reducing cholesterol, comprising the steps:
(a) milling the substantially dry cross-linked polyallylamine salt to a desired particle size,
(b) blending with at least one water soluble excipient,
(c) optionally blending with water after the step (a) or (b), and
(d) optionally blending the mixture obtained in step (c) with an additive and or lubricant.

3. The method of claim 2, wherein step (a) further comprises reducing the particle size of said cross-linked polyallylamine to less than 400 microns.

4. The method of claim 2, further comprising adding water to the cross-linked polyallylamine salt.

5. The composition of claim 1, further comprising at least one lubricant.

6. The composition of claim 1, further comprising at least one additive.

7. The composition of claim 1, further comprising water.

8. The composition of claim 1, wherein the cross-linked polyallylamine salt is sevelamer hydrochloride.

9. The composition of claim 1, wherein the cross-linked polyallylamine salt is sevelamer carbonate.

10. The composition of claim 1, wherein the cross-linked polyallylamine salt is colesevelam hydrochloride.

11. The composition of claim 5, wherein the lubricant is selected from a group consisting of stearic acid, sodium stearate, magnesium stearate, calcium stearate, zinc stearate, or sodium stearylfumarate, or a combination thereof.

12. The composition of claim 6, wherein the additive is selected from a group consisting of silicon dioxide, sodium chloride, or sodium carbonate, or a combination thereof.

13. The composition of claim 1, wherein said composition is directly compressed into a tablet or tablet core.

14. The composition of claim 1, wherein the amount of cross-linked polyallylamine salt is about 60% to 90% by weight.

15. The composition of claim 1, wherein the amount of at least one excipient is about 5% to 35% by weight.

16. The composition of claim 1, wherein said at least one excipient is selected from a group consisting of sorbitol, xylitol, maltitol, Mannitol, dextrose, sucrose, dextrate, Isomalt, maltose, lactitol, maltodextrin, polyethylene glycol, monosaccharide, or disaccharide.

17. The composition of claim 13, wherein said tablet or tablet core is coated.

18. The method of claim 2, wherein said at least one water soluble excipient is selected from the group consisting of sorbitol, xylitol, maltitol, Mannitol, dextrose, sucrose, dextrate, polyethylene glycol, monosaccharide, or disaccharide, or a combination thereof.

19. The method of claim 2, further comprising: blending the mixture of step (a) or step (b) with water.

20. The method of claim 19, further comprising blending the mixture obtained in step (c) with an additive or lubricant, or both.

21. A pharmaceutical composition comprising:
at least one cross-linked polyallylamine salt useful to treat hyperphosphatemia or reducing cholesterol, and at least one water soluble excipient.

22. The method of claim 2, wherein said cross-linked polyallylamine salt has a water content of less than 3%.
